(19) United States
(12) Patent Application Publication
(10) Pub. No.: US 2011/0223220 A1
(43) Pub. Date: Sep. 15, 2011

(54) DISPERSIONS OF ENCAPSULATED PARTICLES AND METHODS FOR THEIR PRODUCTION AND USE

(75) Inventors: Noel R. Vanier, Wexford, PA (US); Cheng-Hung Hung, Wexford, PA (US)

(73) Assignee: PPG Industries Ohio, Inc., Cleveland, OH (US)

(21) Appl. No.: 12/723,829

(22) Filed: Mar. 15, 2010

(51) Int. Cl. A61K 8/04; C11D 17/00; A61Q 19/10; A61Q 17/04; A61Q 11/00; A61Q 15/00; C14C 1/06

(52) U.S. Cl. 424/401; 510/158; 510/119; 424/59; 424/49; 424/65; 8/94.16

(57) ABSTRACT

Disclosed are dispersions of encapsulated particles and methods for their production and use. These dispersions include encapsulated particles and a liquid medium in which the encapsulated particles are dispersed. The encapsulated particles include a carrier particle and an encapsulant deposited on the carrier particle. The liquid medium and the encapsulant are selected so as to be capable of reacting with each other to form a reaction product having a boiling point of no more than 300° C. at atmospheric pressure.

### FIELD OF THE INVENTION

The present invention relates to dispersions of encapsulated particles and methods for making dispersions of non-agglomerated particles, such as ultrafine particles. The present invention also relates to methods for using such dispersions, such as in coatings applications.

### BACKGROUND OF THE INVENTION

Ultrafine particles have become desirable for use in many applications. As the average primary particle size of a material decreases to less than 1 micron a variety of confinement effects can occur that can change the properties of the material. For example, a property can be altered when the entity or mechanism responsible for that property is confined within a space smaller than some critical length associated with that entity or mechanism. As a result, ultrafine particles represent an opportunity for designing and developing a wide range of materials for structural, optical, electronic and chemical applications, such as coatings.

One difficulty with ultrafine particles is agglomeration which can occur during production and/or use of such particles. Agglomeration is a serious problem for ultrafine particles in particular because they have a relatively large surface area. Because it is often desirable, such as in coatings applications, to use such ultrafine particles in the form of a liquid dispersion of such particles in a liquid medium in combination with a resinous grind vehicle and/or dispersant that substantially prevents particle agglomeration, it would be desirable to provide improved methods for making such dispersions.

### SUMMARY OF THE INVENTION

In certain respects, the present invention is directed to dispersions of encapsulated particles in a liquid medium. The encapsulated particles comprising: (a) a carrier particle comprising a surface; and (b) an encapsulant deposited on the surface of the carrier particle. The liquid medium and the encapsulant are selected so as to be capable of reacting with each other to form a reaction product having a boiling point of no more than 300° C. at atmospheric pressure.

In other respects, the present invention is directed to dispersions comprising: (a) encapsulated particles, such as ultrafine particles, comprising a carrier particle comprising a surface and an encapsulant comprising B₂O₃ and/or P₂O₅ deposited on the surface of the carrier particle; and (b) a liquid medium in which the encapsulated particles are dispersed and comprising a hydroxyl containing compound.

In other respects, the present invention is directed to methods for making dispersions of ultrafine particles in a liquid medium. These methods comprise reacting the liquid medium with encapsulated ultrafine particles to form a reaction product that has a boiling point of no more than 300° C. at atmospheric pressure. Thereafter, the reaction product may, if desired, be substantially or completely removed from the dispersion, such removal optionally taking place in the presence of a stabilizing agent, such as a dispersant, so that the resulting ultrafine particles in the dispersion are substantially non-agglomerated.

The present invention also relates to methods for using the dispersions of the present invention, such as in coatings applications.

### BRIEF DESCRIPTION OF THE DRAWINGS

FIG. 1 illustrates an encapsulated particle in accordance with certain embodiments of the present invention.

### DETAILED DESCRIPTION OF EMBODIMENTS OF THE INVENTION

For purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about”. Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances.

As indicated, certain embodiments of the present invention are directed to dispersions of encapsulated particles in a liquid medium. In certain embodiments, the encapsulated particles are ultrafine particles. As used herein, the term “ultrafine particles” refers to particles having a B.E.T. specific surface area of at least 10 square meters per gram, such as 30 to 500 square meters per gram, or, in some cases, 90 to 500 square meters per gram or, in yet other cases, 180 to 500 square meters per gram. As used herein, the term “B.E.T. specific surface area” refers to a specific surface area determined by nitrogen adsorption according to the ASTMD 3663-78 standard based on the Brunauer-Emmett-Teller method described in the periodical “The Journal of the American Chemical Society”, 60, 309 (1938).

In certain embodiments, the ultrafine particles described herein have a calculated equivalent spherical diameter of no more than 200 nanometers, such as no more than 100 nanometers, or, in certain embodiments, 5 to 50 nanometers, or, in yet other cases, 5 to 20 nanometers. As will be understood by those skilled in the art, a calculated equivalent spherical diameter can be determined from the B.E.T. specific surface area according to the following equation:

Diameter (nanometers)=6000/[BET(m²/g)*ρ(grams/cm³)]

In certain embodiments, the ultrafine particles described herein have an average primary particle size of no more than 1000 nanometers, in some cases no more than 500 nanometers, in other cases no more than 400 nanometers, no more than 300 nanometers, no more than 200 nanometers, no more than 100 nanometers, no more than 50 nanometers, no more than 20 nanometers or, in other cases, no more than 12 nanometers. As used herein, the term “primary particle size” refers to a particle size as determined by visually examining a micrograph of a transmission electron microscopy (“TEM”) image, measuring the diameter of the particles in the image, and calculating the average primary particle size of the measured particles based on magnification of the TEM image. One of ordinary skill in the art will understand how to prepare such a TEM image and determine the primary particle size based on the magnification. The primary particle size of a particle refers to the smallest diameter sphere that will completely enclose the particle. As used herein, the term “primary particle size” refers to the size of an individual particle as opposed to an agglomeration of two or more individual particles.

The ultrafine particles described herein may be prepared in any manner well known to those skilled in the art, such as, for example, any gas phase synthesis process, including, for example, flame pyrolysis, hot walled reactor, chemical vapor synthesis, and rapid quench plasma synthesis. Gas phase synthesis processes for producing ultrafine particles are well known and suitable processes are disclosed, for example, in U.S. Pat. Nos. 4,851,262; 5,749,937; 5,788,738; 5,851,507; 5,935,293; 5,984,997; and 6,652,967, among many others. Another specific example of suitable gas phase synthesis processes for producing the ultrafine particles described herein are processes of the type disclosed in U.S. Pat. No. 7,635,458 at col. 3, line 37 to col. 9, line 44, the cited portion of which being incorporated herein by reference. As will be appreciated, in such processes, solid, liquid, and/or gas precursors are introduced to a high temperature chamber, such precursors comprising virtually any kind of material, depending upon the desired composition of the ultrafine particles. The Examples herein are also illustrative.

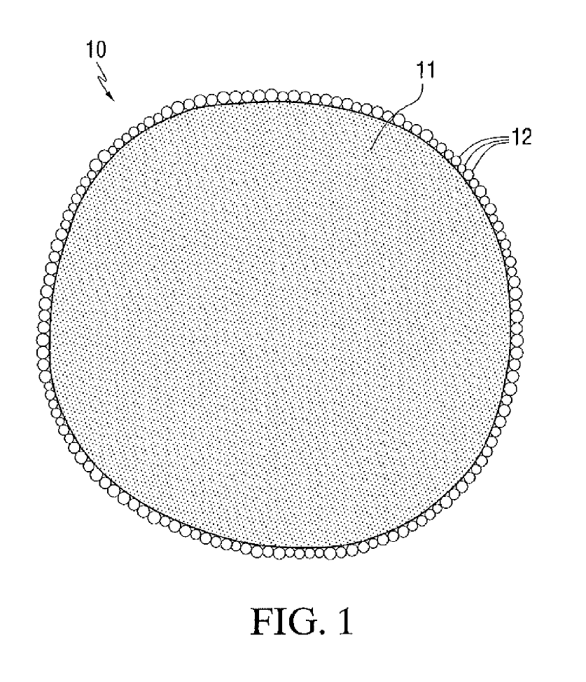

As indicated, the encapsulated particles present in the dispersions of the present invention comprise: (a) a carrier particle comprising a surface; and (b) an encapsulant deposited on the surface of the carrier particle. FIG. 1 schematically illustrates an encapsulated particle in accordance with an embodiment of the present invention. As is apparent, an encapsulated particle 10 comprises a carrier particle 11 and an encapsulant 12, which, in this embodiment, is depicted as a plurality of ultrafine particles. It should be appreciated, however, that the encapsulant need not be in the form of discrete ultrafine particles. In some cases, for example, the encapsulant could be an uncrystallized glass-like solid that covers all or part of the surface of the carrier particle. In certain embodiments, during gas phase synthesis of the ultrafine particles, the relatively large ultrafine carrier particle 11 forms first, followed by heterogeneous nucleation and deposition of encapsulant on the surface of a previously formed carrier particle.

Moreover, although the encapsulant 12 may form a continuous layer on the carrier particle 11, such as is shown in FIG. 1 where adjacent ultrafine particles are depicted as touching each other, in other embodiments the encapsulant may not form a continuous layer, such as could be the case when there is a lower ratio or concentration of the encapsulant 12 in comparison with the carrier particles 11. For example, in certain embodiments, the encapsulant covers at least 1 percent, at least 10 percent, at least 50 percent, at least 70 percent, at least 80 percent, or, in some cases, at least 90 percent, of the entire surface area of the carrier particle. In certain embodiments, the encapsulant covers no more than 99 percent, such no more than 95 percent, of the entire surface area of the carrier particle. In certain embodiments, however, the encapsulant covers 100 percent of the entire surface area of the carrier particle.

Heterogeneous nucleation and deposition of ultrafine encapsulant particles on the surface of a previously formed carrier particle during gas phase synthesis of ultrafine particles can be achieved by methods known in the art, such as, for example, by temperature and partial pressure control of the synthesis process, such as is disclosed in U.S. Pat. No. 5,498,446 at col. 8, line 1 to col. 9, line 4, the cited portion of which being incorporated herein by reference. In certain embodiments, heterogeneous nucleation is achieved by selecting an encapsulant composition that has a boiling point that is less than the melting point of the carrier particle composition (melting points of certain exemplary carrier compositions are provided below), whereas in some embodiments heterogeneous nucleation can be achieved by selecting an encapsulant composition that is incompatible, i.e., will not form a mixed phase, with the selected carrier particle composition. Examplary, but non-limiting, examples of combinations of incompatible compositions, one of which may be selected as a carrier composition and the other as an encapsulant composition in heterogeneous nucleation, are MgO—NaCl, TiO₂—MgF₂, SiO₂—KBr, CaF₂—B₂O₃, SiO₂—Cu, Ti—B₂O₃, CaO—Sn, ZrB₂—B₂O₃, B₄C—B₂O₃, BN—B₂O₃, MgF₂—Zn, SiC—KBr, TiB₂—NaCl, Si₃N₄—CaF₂, and TiB₂—B₄C.

In certain embodiments, the carrier particle 11 has an average primary particle size of no more than 1,000 nanometers, in some cases no more than 500 nanometers, in other cases no more than 400 nanometers, no more than 300 nanometers, no more than 200 nanometers, no more than 100 nanometers. In certain embodiments, the carrier particle has an average particle size of no less than 20 nanometers, in some cases, no less than 50 nanometers. For example, the carrier particles may have an average particle size of from about 100 to about 300 nanometers.

The composition of the carrier particle is not particularly limited and may comprise, for example, a ceramic composition, such as an oxide, a carbide, a nitride, a sulfide, a halide, a boride; or an elemental composition, examples of which are set forth in U.S. Pat. No. 6,652,967 at col. 6, lines 4 to 35, the cited portion of which being incorporated herein by reference. In some embodiments, the ceramic composition for the carrier particle comprises, for example: (a) a simple oxide, such as aluminum oxide (Al₂O₃ has a melting point of 2015° C.), silicon oxide (SiO₂ has a melting point of 1713° C.), zirconium oxide (ZrO₂ has a melting point of 2700° C.), titanium oxide (TiO₂ has a melting point of 1830° C.), magnesium oxide (MgO has a melting point of 2800° C.), calcium oxide (CaO has a melting point of 2580° C.), and/or copper oxide (Cu₂O has a melting point of 1235° C.); (b) a multi-metal oxide, such as zinc silicon oxide (ZnSiO₃ has a melting point of 1437° C.); (c) a carbide such as silicon carbide (SiC has a melting point of 2830° C.), and/or boron carbide (B₄C has a melting point of 2350° C.); (d) a nitride, such as silicon nitride (Si₃N₄ has a melting point of 1900° C.); (e) a boride, such as titanium diboride (melting point of 2900° C.) and/or tungsten diboride (melting point of 2900° C.); (f) a sulfide, such as zinc sulfide (ZnS has a melting point of 1185° C.); and/or (g) a halide, such as calcium fluoride (CaF₂ has a melting point of 1360° C.), magnesium fluoride (MgF₂ has a melting point of 1266° C.), and/or sodium chloride (NaCl has a melting point of 801° C.). In some embodiments, the elemental composition for the carrier particle comprises, for example, copper (melting point of 1083° C.), titanium (melting point of 1675° C.), boron (melting point of 2300° C.), and/or silicon (melting point of 1410° C.).

In certain embodiments of the liquid dispersions of the present invention, it is desirable that the carrier particle comprise a composition that is not capable of reacting with the liquid medium to form a reaction product having a boiling point, at atmospheric pressure, of no more than 300° C.

In certain embodiments, the encapsulant 12 comprises ultrafine particles having an average particle size of no more than 20 nanometers, such as no more than 10 nanometers. In certain embodiments, the encapsulant comprises ultrafine particles having an average particle size of from 1 to 5 nanometers, such as 2 to 4 nanometers.

In the liquid dispersions of the present invention, the encapsulant comprises a composition capable of reacting with the liquid medium to form a reaction product having a boiling point of no more than 300° C., in some cases no more than 200° C., or, in yet other cases, no more than 100° C., or, in yet other cases, no more than 70° C., at atmospheric pressure. The composition of the encapsulant is not limited so long as it is capable of reacting with the liquid medium to form such a reaction product. As a result, the carrier particle may comprise, for example, a ceramic and/or elemental composition, including certain of those described earlier with respect to the carrier particle.

In certain embodiments, however, the encapsulant comprises an oxoacid (an acid in which the acidic hydrogen is part of a hydroxyl group bound to an atom that is bound to an oxo group (═O)), examples of which include B₂O₃ and/or P₂O₅, that is capable of reacting with a liquid medium comprising a hydroxyl containing compound, such as, for example, an alcohol and/or a phenol, to form an ester, e.g., a borate or phosphate. As will be appreciated, B₂O₃ and P₂O₅ are each capable of reacting with certain alcohols, such as relatively low molecular weight C₁-C₄ monoalcohols, including methanol, ethanol, isopropanol, n-propanol, isobutanol, t-butanol, and/or n-butanol, to form water and a borate or phosphate that has a boiling point of no more than 300° C. By way of a few specific examples, B₂O₃ is capable of reacting with (i) methanol to form water and trimethyl borate, which has a boiling point at atmospheric pressure of less than 70° C.; (ii) ethanol to form water and triethyl borate, which has a boiling point at atmospheric pressure of about 118° C.; (iii) n-propanol to form water and tripropyl borate, which has a boiling point at atmospheric pressure of about 180° C.; (iv) isopropanol to form water and triisopropyl borate, which has a boiling point at atmospheric pressure of about 104° C.; (v) n-butanol to form water and tributyl borate, which has a boiling point at atmospheric pressure of about 232° C. In addition, P₂O₅ is capable of reacting with (i) methanol to form water and trimethyl phosphate, which has a boiling point at atmospheric pressure of about 197° C.; (ii) ethanol to form water and triethyl phosphate, which has a boiling point at atmospheric pressure of about 216° C.; (iii) n-propanol to form water and tripropyl phosphate, which has a boiling point at atmospheric pressure of about 252° C.; (iv) isopropanol to form water and triisopropyl phosphate, which has a boiling point at atmospheric pressure of about 220° C.; (v) n-butanol to form water and tributyl phosphate, which has a boiling point at atmospheric pressure of about 289° C.; and (vi) iso-butanol to form water and triisobutyl phosphate, which has a boiling point at atmospheric pressure of about 264° C.

In addition to the aforementioned materials, the dispersions of the present invention may include other components. For example, the dispersion may comprise a diluent so that the dispersion will have a desired viscosity. Suitable diluents include, for example, water and any of a variety of organic solvents, including ketones, such as methyl ethyl ketone, methyl isobutyl ketone and isophorone; esters and ethers, such as 2-ethoxyethyl acetate and 2-ethoxyethanol; aromatic hydrocarbons, such as benzene, toluene, and xylene; and aromatic solvent blends derived from petroleum, such as those sold commercially under the trademark SOLVESSO®. The amount of diluent will vary depending on the desired viscosity of the dispersion.

In certain embodiments, the dispersions of the present invention comprise a stabilizing agent, i.e., a dispersant that prevents, or substantially prevents, agglomeration of the ultrafine particles upon removal of the encapsulant. Suitable stabilizing agents include, for example, any of the dispersants described in Kirk Othmer Encyclopedia of Chemical Technology, Fifth Edition, Volume 8, pp. 672-697, which description is herein incorporated by reference.

As indicated, in certain embodiments, the encapsulant and the liquid medium are reacted to form a reaction product that has a boiling point of no more than 300° C., in some cases no more than 200° C., or, in yet other cases, no more than 100° C., or, in yet other cases, no more than 70° C., at atmospheric pressure. In certain embodiments, such a reaction can be caused to occur through mild heating, such as is described in the Examples. As a result, the present invention is also directed to methods for making dispersions of ultrafine particles in a liquid medium. These methods comprise reacting the liquid medium with encapsulated ultrafine particles to form a reaction product that has a boiling point of no more than 300° C. at atmospheric pressure.

As indicated earlier, the reaction product may, if desired, be substantially or completely removed from the dispersion, such removal optionally taking place in the presence of a stabilizing agent, such as a dispersant, so that the resulting ultrafine particles in the dispersion remain substantially non-agglomerated. Removal of the reaction product can take place, for example, by heating the dispersion to a temperature greater than the boiling point of the reaction product. Again, the Examples herein are illustrative. By “substantially non-agglomerated” it is meant that the measured average particle diameter is within a factor of three of the average primary particle diameter. Aggregate particle diameter is typically measured using light scattering techniques known in the art. Primary particle diameter is typically measured using BET and TEM, as described above.

The present invention is also directed to methods for using the dispersions described herein, particularly the dispersions of substantially non-agglomerated ultrafine particles resulting from the above described methods. These methods comprise including the dispersion as part of a larger composition such as, for example, those compositions suitable for application to at least a portion of a surface of an object, i.e., a substrate. Objects to which the compositions of the present invention may be applied include animate objects, i.e., living beings, and inanimate objects, including both naturally occurring and man-made objects.

Examples of animate objects to which the compositions of the present invention may be applied include plants and animals, including human beings. For example, the dispersions of the present invention may be employed in compositions that are applied to various human and/or animal substrates, such as keratin, fur, skin, teeth, nails, and the like.

As a result, in certain embodiments, the dispersions of the present invention are employed in personal care products, including, for example, bath and shower gels, shampoos, conditioners, cream rinses, hair dyes, leave-on conditioners, sunscreens, sun tan lotions, body bronzers, and sunblocks, lip balms, skin conditioners, hair sprays, soaps, body scrubs, exfoliants, astringents, depilatories and permanent waving solutions, antidandruff formulations, antisweat and antiperspirant compositions, shaving, preshaving and after shaving products, moisturizers, mouthwashes, toothpastes, deodorants, cold creams, cleansers, skin gels, rinses, whether in solid, powder, liquid, cream, paste, gel, ointment, lotion, emulsions, colloids, solutions, suspensions, or other form.

In other embodiments, the dispersions of the present invention are included in cosmetic compositions, including, without limitation, lipstick, mascara, rouge, foundation, blush, eyeliner, lipliner, lip gloss, facial or body powder, sunscreens and blocks, nail polish, mousse, sprays, styling gels, nail conditioner, whether in the form of creams, lotions, gels, ointments, emulsions, colloids, solutions, suspensions, compacts, solids, pencils, spray-on formulations, brush-on formulations and the like.

In yet other embodiments, the dispersions of the present invention are employed in pharmaceutical preparations including, without limitation, carriers for dermatological purposes, including topical and transdermal application of pharmaceutically active ingredients. These can be in the form of gels, pastes, patches, creams, nose sprays, ointments, lotions, emulsions, colloids, solutions, suspensions, powders and the like.

In certain embodiments, the dispersions of the present invention are employed in coating compositions that comprise a film-forming resin. As used herein, the term “film-forming resin” refers to resins that can form a self-supporting continuous film on at least a horizontal surface of a substrate upon removal of any diluents or carriers present in the composition or upon curing at ambient or elevated temperature.

Film-forming resins that may be used in the coating compositions of the present invention include, without limitation, those used in automotive OEM coating compositions, automotive refinish coating compositions, industrial coating compositions, architectural coating compositions, coil coating compositions, and aerospace coating compositions, among others.

In certain embodiments, the film-forming resin included within the coating compositions of the present invention comprises a thermosetting film-forming resin. As used herein, the term “thermosetting” refers to resins that “set” irreversibly upon curing or crosslinking, wherein the polymer chains of the polymeric components are joined together by covalent bonds. This property is usually associated with a cross-linking reaction of the composition constituents often induced, for example, by heat or radiation. See Hawley, Gessner G., The Condensed Chemical Dictionary, Ninth Edition., page 856; Surface Coatings, vol. 2, Oil and Colour Chemists' Association, Australia, TAFE Educational Books (1974). Curing or crosslinking reactions also may be carried out under ambient conditions. Once cured or crosslinked, a thermosetting resin will not melt upon the application of heat and is insoluble in solvents. In other embodiments, the film-forming resin included within the coating compositions of the present invention comprises a thermoplastic resin. As used herein, the term “thermoplastic” refers to resins that comprise polymeric components that are not joined by covalent bonds and thereby can undergo liquid flow upon heating and are soluble in solvents. See Saunders, K. J., Organic Polymer Chemistry, pp. 41-42, Chapman and Hall, London (1973).

Film-forming resins suitable for use in the coating compositions of the present invention include, for example, those formed from the reaction of a polymer having at least one type of reactive group and a curing agent having reactive groups reactive with the reactive group(s) of the polymer. As used herein, the term “polymer” is meant to encompass oligomers, and includes, without limitation, both homopolymers and copolymers. The polymers can be, for example, acrylic, saturated or unsaturated polyester, polyurethane or polyether, polyvinyl, cellulosic, acrylate, silicon-based polymers, co-polymers thereof, and mixtures thereof, and can contain reactive groups such as epoxy, carboxylic acid, hydroxyl, isocyanate, amide, carbamate and carboxylate groups, among others, including mixtures thereof.

Suitable acrylic polymers include, for example, those described in United States Patent Application Publication 2003/0158316 A1 at [0030]-[0039], the cited portion of which being incorporated herein by reference. Suitable polyester polymers include, for example, those described in United States Patent Application Publication 2003/0158316 A1 at [0040]-[0046], the cited portion of which being incorporated herein by reference. Suitable polyurethane polymers include, for example, those described in United States Patent Application Publication 2003/0158316 A1 at [0047]-[0052], the cited portion of which being incorporated herein by reference. Suitable silicon-based polymers are defined in U.S. Pat. No. 6,623,791 at col. 9, lines 5-10, the cited portion of which being incorporated herein by reference.

As indicated earlier, certain coating compositions of the present invention can include a film-forming resin that is formed from the use of a curing agent. As used herein, the term “curing agent” refers to a material that promotes “cure” of composition components. As used herein, the term “cure” means that any crosslinkable components of the composition are at least partially crosslinked. In certain embodiments, the crosslink density of the crosslinkable components, i.e., the degree of crosslinking, ranges from 5 percent to 100 percent of complete crosslinking, such as 35 percent to 85 percent of complete crosslinking. One skilled in the art will understand that the presence and degree of crosslinking, i.e., the crosslink density, can be determined by a variety of methods, such as dynamic mechanical thermal analysis (DMTA) using a Polymer Laboratories MK III DMTA analyzer, as is described in U.S. Pat. No. 6,803,408, at col. 7, line 66 to col. 8, line 18, the cited portion of which being incorporated herein by reference.

Any of a variety of curing agents known to those skilled in the art may be used. For example exemplary suitable aminoplast and phenoplast resins are described in U.S. Pat. No. 3,919,351 at col. 5, line 22 to col. 6, line 25, the cited portion of which being incorporated herein by reference. Exemplary suitable polyisocyanates and blocked isocyanates are described in U.S. Pat. No. 4,546,045 at col. 5, lines 16 to 38; and in U.S. Pat. No. 5,468,802 at col. 3, lines 48 to 60, the cited portions of which being incorporated herein by reference. Exemplary suitable anhydrides are described in U.S. Pat. No. 4,798,746 at col. 10, lines 16 to 50; and in U.S. Pat. No. 4,732,790 at col. 3, lines 41 to 57, the cited portions of which being incorporated herein by reference. Exemplary suitable polyepoxides are described in U.S. Pat. No. 4,681,811 at col. 5, lines 33 to 58, the cited portion of which being incorporated herein by reference. Exemplary suitable polyacids are described in U.S. Pat. No. 4,681,811 at col. 6, line 45 to col. 9, line 54, the cited portion of which being incorporated herein by reference. Exemplary suitable polyols are described in U.S. Pat. No. 4,046,729 at col. 7, line 52 to col. 8, line 9; col. 8, line 29 to col. 9, line 66; and in U.S. Pat. No. 3,919,315 at col. 2, line 64 to col. 3, line 33, the cited portions of which being incorporated herein by reference. Examples suitable polyamines described in U.S. Pat. No. 4,046,729 at col. 6, line 61 to col. 7, line 26, and in U.S. Pat. No. 3,799,854 at column 3, lines 13 to 50, the cited portions of which being incorporated herein by reference. Appropriate mixtures of curing agents, such as those described above, may be used.

In certain embodiments, the film-forming resin is present in the coating compositions of the present invention in an amount greater than 30 weight percent, such as 40 to 90 weight percent, or, in some cases, 50 to 90 weight percent, with weight percent being based on the total weight of the coating composition. When a curing agent is used, it may, in certain embodiments, be present in an amount of up to 70 weight percent, such as 10 to 70 weight percent; this weight percent is also based on the total weight of the coating composition.

In certain embodiments, the coating compositions of the present invention are in the form of liquid coating compositions, examples of which include aqueous and solvent-based coating compositions and electrodepositable coating compositions. The coating compositions of the present invention may also be in the form of a co-reactable solid in particulate form, i.e., a powder coating composition. Regardless of the form, the coating compositions of the present invention may be pigmented or clear, and may be used alone or in combination as primers, basecoats, or topcoats.

In certain embodiments, the coating compositions of the present invention may also comprise additional optional ingredients, such as those ingredients well known in the art of formulating surface coatings. Such optional ingredients may comprise, for example, surface active agents, flow control agents, thixotropic agents, fillers, anti-gassing agents, organic co-solvents, catalysts, antioxidants, light stabilizers, UV absorbers and other customary auxiliaries. Any such additives known in the art can be used, absent compatibility problems. Non-limiting examples of these materials and suitable amounts include those described in U.S. Pat. Nos. 4,220,679; 4,403,003; 4,147,769; and 5,071,904. The coating compositions of the present invention can also include a colorant and/or corrosion resisting particles, such as, for example, any of those disclosed in United States Patent Application Publication No. 2008/0075649 A1 at [0069] to [0079], the cited portion of which being incorporated herein by reference.

As should also be apparent from the foregoing description, the present invention is also directed to methods for reducing the average primary particle size of ultrafine particles. Such methods comprise: (a) reacting a liquid medium with encapsulated ultrafine particles comprising (i) a carrier ultrafine particle comprising a surface; and (ii) an encapsulant deposited on the surface of the ultrafine particle, wherein the reaction forms a reaction product that has a boiling point of no more than 300° C. at atmospheric pressure; and (b) removing the reaction product from the dispersion. If desired, the dispersion can be dried to provide dry ultrafine particles without the encapsulant.

Illustrating the invention are the following examples, which, however, are not to be considered as limiting the invention to their details. Unless otherwise indicated, all parts and percentages in the following examples, as well as throughout the specification, are by weight.

### EXAMPLES

### Example 1

Ultrafine boron carbide particles were produced using a DC thermal plasma reactor system. The main reactor system included a DC plasma torch (Model SG-100 Plasma Spray Gun commercially available from Praxair Technology, Inc., Danbury, Conn.) operated with 60 standard liters per minute of argon carrier gas and 24 kilowatts of power delivered to the torch. A liquid precursor feed composition comprising the materials and amounts listed in Table 1 was prepared and fed to the reactor at a rate of 7 grams per minute through a gas assisted liquid nebulizer located about 0.5 inch down stream of the plasma torch outlet. At the nebulizer, 15 standard liters per minute of argon were delivered to assist in atomization of the liquid precursors. Following a 10 inch long reactor section, a plurality of quench stream injection ports were provided that included 6⅛ inch diameter nozzles located 60° apart radially. A 7 millimeter diameter converging-diverging nozzle was provided 4 inches downstream of the quench stream injection port. Quench argon gas was injected through the quench stream injection ports at a rate of 145 standard liters per minute.

TABLE 1

| Material | Amount |
|---|---|
| Trimethyl Borate¹ | 1000 grams |
| Iso-Octane¹ | 33.7 grams |

¹Commercially available from Alfa Aesar, Ward Hill, Massachusetts.

The measured B.E.T. specific surface area of the produced material was 21 square meters per gram using a Gemini model 2360 analyzer (available from Micromeritics Instrument Corp., Norcross, Ga.), and the calculated equivalent spherical diameter was 113 nanometers.

The produced powder was washed using methanol and toluene to remove boron oxide encapsulant. For every 100 grams raw powder, 100 grams methanol and 60 grams toluene were added. The dispersion was heated up to about 60 degree centigrade for reactions. Later, the dispersion was heated up to 110 degree centigrade to distill off all solvent. In the second wash, additional 100 grams methanol was added to continue to remove boron oxide residue. The dispersion was heated up to 60 degree centigrade for reaction and then heated up to 110 degree centigrade to remove all solvent. The addition of methanol and heating cycle were repeated additional 4 times. After 6^{th} wash was completed, a full vacuum was applied to the system for 45 minutes to dry up the powder. The collected powder was then further dried up in a vacuum oven at 115 degree centigrade for one hour.

The measured B.E.T. specific surface area of the washed material was 50 square meters per gram using a Gemini model 2360 analyzer (available from Micromeritics Instrument Corp., Norcross, Ga.), and the calculated equivalent spherical diameter was 48 nanometers.

The reduction in particle size before and after the washing process was indicative of the presence of a B₂O₃ on the pre-washed ultrafine particles.

### Example 2

Ultrafine boron carbide particles from nitrogen-containing liquid precursors were prepared using the apparatus and conditions identified in Example 1, with the feed materials and amounts listed in Table 2.

TABLE 2

| Material | Amount |
|---|---|
| Trimethyl Borate | 1000 grams |
| N,N-Dimethyl Formamide¹ | 86.1 grams |

The measured B.E.T. specific surface area of the produced material was 22 square meters per gram using a Gemini model 2360 analyzer (available from Micromeritics Instrument Corp., Norcross, Ga.), and the calculated equivalent spherical diameter was 108 nanometers.

The produced raw boron carbide particles were purified using the apparatus and conditions identified in Example 1. The measured B.E.T. specific surface area of the washed material was 33 square meters per gram using a Gemini model 2360 analyzer (available from Micromeritics Instrument Corp., Norcross, Ga.), and the calculated equivalent spherical diameter was 72 nanometers.

The reduction in particle size before and after the washing process was indicative of the presence of a B₂O₃ on the pre-washed ultrafine particles.

### Example 3

Boron oxide encapsulated silica particles were prepared using a DC thermal plasma system. The plasma system included a DC plasma torch (Model SG-100 Plasma Spray Gun commercially available from Praxair Technology, Inc., Danbury, Conn.) operated with 60 standard liters per minute of argon carrier gas and 16 kilowatts of power delivered to the torch. A solid precursor feed composition comprising the materials and amounts listed in Table 3 was prepared and fed to the reactor at a rate of about 1 grams per minute through a gas assistant powder feeder (Model 1264 commercially available from Praxair Technology) located at the plasma torch outlet. At the powder feeder, 4.7 standard liters per minute argon was delivered as a carrier gas. Argon was delivered at 5 standard liters per minute through two ⅛ inch diameter nozzles located 180° apart at 0.69 inch downstream of the powder injection port. Following a 9.7 inch long reactor section, a plurality of quench stream injection ports were provided that included 6⅛ inch diameter nozzles located 60° apart radially. A 7 millimeter diameter converging-diverging nozzle of the type described in U.S. Pat. No. RE 37,853E was located 3 inches downstream of the quench stream injection ports. Argon quench gas was injected through the plurality of at the quench stream injection ports at a rate of 145 standard liters per minute.

TABLE 3

| Material | Amount |
|---|---|
| Boron Oxide¹ | 10 grams |
| Silica² | 90 grams |

¹Commercially available from Alfa Aesar Co., Ward Hill, MA.
²Commercially available under the tradename WB-10 from PPG Industries, Inc., Pittsburgh, PA.

The measured B.E.T. specific surface area of the produced material was 186 square meters per gram using a Gemini model 2360 analyzer (available from Micromeritics Instrument Corp., Norcross, Ga.), and the calculated equivalent spherical diameter was 15 nanometers.

The produced raw boron carbide particles were purified using the apparatus and conditions identified in Example 1. The measured B.E.T. specific surface area of the washed material was 221 square meters per gram using a Gemini model 2360 analyzer (available from Micromeritics Instrument Corp., Norcross, Ga.), and the calculated equivalent spherical diameter was 12 nanometers.

The reduction in particle size before and after the washing process was indicative of the presence of a B₂O₃ on the pre-washed ultrafine particles.

### Example 4

Boron oxide encapsulated silica particles were prepared using the apparatus and conditions identified in Example 3, with the feed materials and amounts listed in Table 4.

TABLE 4

| Material | Amount |
|---|---|
| Boron Oxide¹ | 30 grams |
| Silica² | 70 grams |

¹Commercially available from Alfa Aesar Co., Ward Hill, MA.
²Commercially available under the tradename WB-10 from PPG Industries, Inc., Pittsburgh, PA.

The measured B.E.T. specific surface area of the produced material was 77 square meters per gram using a Gemini model 2360 analyzer (available from Micromeritics Instrument Corp., Norcross, Ga.), and the calculated equivalent spherical diameter was 34 nanometers.

The produced raw boron carbide particles were purified using the apparatus and conditions identified in Example 1. The measured B.E.T. specific surface area of the washed material was 220 square meters per gram using a Gemini model 2360 analyzer (available from Micromeritics Instrument Corp., Norcross, Ga.), and the calculated equivalent spherical diameter was 12 nanometers.

The reduction in particle size before and after the washing process was indicative of the presence of a B₂O₃ on the pre-washed ultrafine particles.

### Example 5

Boron oxide encapsulated silica particles were prepared using the apparatus and conditions identified in Example 3, with the feed materials and amounts listed in Table 5.

TABLE 5

| Material | Amount |
|---|---|
| Boron Oxide¹ | 100 grams |
| Silica² | 100 grams |

¹Commercially available from Alfa Aesar Co., Ward Hill, MA.
²Commercially available under the tradename WB-10 from PPG Industries, Inc., Pittsburgh, PA.

The measured B.E.T. specific surface area of the produced material was 27 square meters per gram using a Gemini model 2360 analyzer (available from Micromeritics Instrument Corp., Norcross, Ga.), and the calculated equivalent spherical diameter was 96 nanometers.

The produced raw boron carbide particles were purified using the apparatus and conditions identified in Example 1. The measured B.E.T. specific surface area of the washed material was 180 square meters per gram using a Gemini model 2360 analyzer (available from Micromeritics Instrument Corp., Norcross, Ga.), and the calculated equivalent spherical diameter was 15 nanometers.

The reduction in particle size before and after the washing process was indicative of the presence of a B₂O₃ on the pre-washed ultrafine particles.

It will be readily appreciated by those skilled in the art that modifications may be made to the invention without departing from the concepts disclosed in the foregoing description. Such modifications are to be considered as included within the following claims unless the claims, by their language, expressly state otherwise. Accordingly, the particular embodiments described in detail herein are illustrative only and are not limiting to the scope of the invention which is to be given the full breadth of the appended claims and any and all equivalents thereof.

We claim:

1. A dispersion comprising:
(a) encapsulated particles comprising:
(i) a carrier particle comprising a surface; and
(ii) an encapsulant deposited on the surface of the carrier particle; and
(b) a liquid medium in which the encapsulated particles are dispersed,
wherein the liquid medium and the encapsulant are selected so as to be capable of reacting with each other to form a reaction product having a boiling point of no more than 300° C. at atmospheric pressure.

2. The dispersion of claim 1, wherein the encapsulant comprises ultrafine particles.

3. The dispersion of claim 1, wherein the encapsulant forms a continuous layer on the carrier particle.

4. The dispersion of claim 1, wherein the carrier particle has a particle size of no more than 400 nanometers.

5. The dispersion of claim 1, wherein the carrier particle comprises an oxide.

6. The dispersion of claim 5, wherein the oxide comprises SiO₂.

7. The dispersion of claim 1, wherein the carrier particle comprises a carbide.

8. The dispersion of claim 7, wherein the carbide comprises B₄C.

9. The dispersion of claim 1, wherein the reaction product has a boiling point of no more than 200° C. at atmospheric pressure.

10. The dispersion of claim 9, wherein the reaction product has a boiling point of no more than 70° C. at atmospheric pressure.

11. The dispersion of claim 1, wherein the encapsulant comprises an oxoacid.

12. The dispersion of claim 11, wherein the encapsulant comprises B₂O₃ and/or P₂O₅.

13. The dispersion of claim 11, wherein the liquid medium comprises a hydroxyl containing compound.

14. The dispersion of claim 13, wherein the hydroxyl containing compound comprises a C₁-C₄ monoalcohol.

15. The dispersion of claim 1, further comprising a stabilizing agent.

16. A dispersion comprising:
(a) encapsulated ultrafine particles comprising:
(i) a carrier particle comprising a surface; and
(ii) an encapsulant comprising B₂O₃ and/or P₂O₅ deposited on the surface of the carrier particle; and
(b) a liquid medium in which the encapsulated particles are dispersed and comprising a C₁-C₄ monoalcohol.

17. A method for making a dispersion of ultrafine particles in a liquid medium, the method comprising reacting a liquid medium with encapsulated ultrafine particles to form a reaction product that has a boiling point of no more than 300° C. at atmospheric pressure.

18. The method of claim 17, wherein the reaction takes place in the presence of a stabilizing agent.

19. The method of claim 17, further comprising removing the reaction product from the dispersion.

20. The method of claim 17, wherein the encapsulated ultrafine particles comprise an encapsulant comprising B₂O₃ and/or P₂O₅ and the liquid medium comprises a C₁-C₄ monoalcohol.
